(19) United States
(12) Patent Application Publication
(10) Pub. No.: US 2007/0077614 A1
(43) Pub. Date: Apr. 5, 2007

(54) Uses of lp-pla2 in combination to assess coronary risk

(76) Inventors: Robert L. Wolfert, Palo Alto, CA (US); Yu Ping Maguire, San Francisco, CA (US); Yu Ping Li, Fremont, CA (US); Mark Joseph Sarno, Encinitas, CA (US)

Correspondence Address: LICATA & TYRRELL P.C., 66 E. MAIN STREET, MARLTON, NJ, 08053, US

(21) Appl. No.: 10/552,084

(22) PCT Filed: Apr. 1, 2004
(86) PCT No.: PCT/US04/10039

§ 371 Date: Dec. 1, 2006

Related U.S. Application Data
(60) Provisional application No. 60/459,785, filed on Apr. 1, 2003.

(51) Int. Cl. C12Q 1/34

(52) U.S. Cl. 435/18

(57) ABSTRACT

This invention relates to a method for assessing risk of Coronary Vascular Disease (CVD). Specifically, it relates to utilizing risk assessment from both Lipoprotein Associated Phospholipase A2 (Lp-PLA2) and C-reactive protein (CRP) in combination. In addition the invention relates to a method for assessing risk of Coronary Vascular Disease (CVD) in a patient with low to normal Low Density Lipoprotein Cholesterol (LDL) levels utilizing both LDL and Lipoprotein Associated Phospholipase A2 (Lp-PLA2). Moreover, the invention relates to the use of risk associated with Lp-PLA2, CRP and LDL in combination and specific ranges thereof to predict Coronary Vascular Disease.

### FIELD OF THE INVENTION

This invention relates to a method for assessing risk of Coronary Vascular Disease (CVD). Specifically, it relates to utilizing risk assessment from both Lipoprotein Associated Phospholipase A2 (Lp-PLA2) and C-reactive protein (CRP) in combination. In addition the invention relates to a method for assessing risk of Coronary Vascular Disease (CVD) in a patient with low to normal Low Density Lipoprotein Cholesterol (LDL) levels utilizing both LDL and Lipoprotein Associated Phospholipase A2 (Lp-PLA2). Moreover, the invention relates to the use of risk associated with Lp-PLA2, CRP and LDL in combination and specific ranges thereof to predict Coronary Vascular Disease.

### BACKGROUND OF THE INVENTION

Introduction

Coronary heart disease (CHD) is the single most prevalent fatal disease in the United States. In the year 2003, an estimated 1.1 million Americans are predicted to have a new or recurrent coronary attack (see the American Heart Association web site). Approximately 60% of these individuals have no previously known risk factors.

While research continues to link elevated LDL-cholesterol levels with CHD risk, it is well understood that a significant number of individuals with normal LDL-cholesterol levels experience a cardiac event (Eaton 1998), suggesting that other factors not currently recognized may be involved. In the search for new risk factors, significant attention has been focused in recent years on markers of inflammation, as a growing body of basic and clinical research emerges regarding the role of inflammation in atherogenesis (Lusis 2000, Lindahl 2000). Some of the inflammatory markers under investigation include cell adhesion molecules, CD-40 ligand, interleukin 6 and C-reactive protein (CRP). CRP, a non-specific acute phase inflammatory marker, has recently received significant attention as a potential risk indicator for CHD (Ridker 2002, Blake 2002). CRP, however, is well known to be responsive to any source of inflammation, which justifies further investigations to identify more specific markers of arterial involvement.

In preliminary studies, lipoprotein-associated phospholipase A2 (Lp-PLA2) levels have been shown to be significantly correlated in men with angiographically-proven CHD (Caslake 2000) and associated with cardiac events in men with hypercholesterolemia (Packard 2000).

Previously, various methods for detecting Lp-PLA2 have been reported which include immunoassays (Caslake, M. J., C. J. Packard, et al. (2000). Lipoprotein-associated phospholipase A(2), platelet-activating factor acetylhydrolase: a potential new risk factor for coronary artery disease. Atherosclerosis 150(2): 413-9) and activity assays (PAF Acetylhydrolase Assay Kit, Cat#760901 product brochure, Cayman Chemical, Ann Arbor, MI, Dec. 18, 1997 (www.caymanchem.com); Azwell Auto PAF-AH Assay Kit, product instruction manual, Karlan Research Products Corp, Santa Rosa, Calif. (www.karlan.com) announced Jun. 16, 2002; Kosaka, T. et al., Spectrophotometric assay for serum platelet-activating factor acetylhydrolase activity. Clinica Chimica Acta 296 (2000):151-161; Tselepis, A. D. et al., PAF-Degrading Acetylhydrolase is Preferentially Associated with Dense LDL and VHDL-1 in Human Plasma. Arter. Throm. And Vasc. Biol. (1995)15:1764-1773; Kujiraoka T. et al., Altered distribution of plasma PAF-AH between HDLs and other lipoproteins in hyperlipidemia and diabetes mellitus. J Lipid Res. 2003 October;44(10):2006-14). Additionally, the United States Food and Drug Administration (FDA) has granted approval for an ELISA test for the quantitative determination of Lp-PLA2 in human plasma to be used as a predictor of risk for coronary heart disease (CHD) ((2003) September-October; New test predicts heart risk. FDA Consum. 37(5):6.).

Antibodies used in immunoassays may be labeled with an enzyme for detection. Typical substrates for the enzymes for production and deposition of visually detectable products include o nitrophenyl beta D galactopyranoside (ONPG); o phenylenediamine dihydrochloride (OPD); p nitrophenyl phosphate (PNPP); p-nitrophenyl-beta-D-galactopryanoside (PNPG); 3′,3′-diaminobenzidine (DAB); 3-amino-9-ethylcarbazole (AEC); 4 chloro 1 naphthol (CN); 5 bromo 4 chloro 3 indolyl phosphate (BCIP); ABTS®; BluoGal; iodonitrotetrazolium (INT); nitroblue tetrazolium chloride (NBT); phenazine methosulfate (PMS); phenolphthalein monophosphate (PMP); tetramethyl benzidine (TMB); tetranitroblue tetrazolium (TNBT); X Gal; X Gluc; and X Glucoside.

Other substrates can be used to produce products for local deposition that are luminescent. For example, in the presence of hydrogen peroxide (H2O2), horseradish peroxidase (HRP) can catalyze the oxidation of cyclic diacylhydrazides, such as luminol. Immediately following the oxidation, the luminol is in an excited state (intermediate reaction product), which decays to the ground state by emitting light. Strong enhancement of the light emission is produced by enhancers, such as phenolic compounds. Advantages include high sensitivity, high resolution, and rapid detection without radioactivity and requiring only small amounts of antibody. See, e.g., Thorpe et al., Methods Enzymol. 133: 331 53 (1986); Kricka et al., J. Immunoassay 17(1): 67 83 (1996); and Lundqvist et al., J. Biolumin. Chemilumin. 10(6): 353 9 (1995). Kits for such enhanced chemiluminescent detection (ECL) are available commercially. The antibodies can also be labeled using colloidal gold.

As another example, when the antibodies of the present invention are used, e.g., for flow cytometric detection, for scanning laser cytometric detection, or for fluorescent immunoassay, they can usefully be labeled with fluorophores. There are a wide variety of fluorophore labels that can usefully be attached to the antibodies of the present invention. For flow cytometric applications, both for extracellular detection and for intracellular detection, common useful fluorophores can be fluorescein isothiocyanate (FITC), allophycocyanin (APC), R-phycoerythrin (PE), peridinin chlorophyll protein (PerCP), Texas Red, Cy3, Cy5, fluorescence resonance energy tandem fluorophores such as PerCP-Cy5.5, PE-Cy5, PE-Cy5.5, PE-Cy7, PE-Texas Red, and APC-Cy7.

Other fluorophores include, inter alia, Alexa Fluor® 350, Alexa Fluor® 488, Alexa Fluor® 532, Alexa Fluor® 546, Alexa Fluor® 568, Alexa Fluor® 594, Alexa Fluor® 647 (monoclonal antibody labeling kits available from Molecular Probes, Inc., Eugene, Oreg., USA), BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA), and Cy2, Cy3, Cy3.5, Cy5, Cy5.5, Cy7, all of which are also useful for fluorescently labeling the antibodies of the present invention. For secondary detection using labeled avidin, streptavidin, captavidin or neutravidin, the antibodies of the present invention can usefully be labeled with biotin.

When the antibodies of the present invention are used, e.g., for western blotting applications, they can usefully be labeled with radioisotopes, such as 33P, 32P, 35S, 3H, and 125I. As another example, when the antibodies of the present invention are used for radioimmunotherapy, the label can usefully be 3H, 228Th, 227Ac, 225Ac, 223Ra, 213Bi, 212Pb, 212Bi, 211At, 203Pb, 194Os, 188Re, 186Re, 153Sm, 149Tb, 131I, 125I, 111In, 105Rh, 99mTc, 97Ru, 90Y, 90Sr, 88Y, 72Se, 67Cu, or 47Sc.

Background Information on Coronary Heart Disease

Coronary vascular disease (CVD) encompasses all diseases of the vasculature, including high blood pressure, CHD, stroke, congenital cardiovascular defects and congestive heart failure. Studies have shown that CHD is responsible for the majority of the CVD. The prevalence of CHD increases markedly as a function of age, with men having a higher prevalence than women within most age groups.

The current standard of care used to identify individuals at risk for heart disease is the measurement of a lipid panel, including triglycerides, total cholesterol, low density lipoprotein (LDL)-cholesterol, and high density lipoprotein (HDL)-cholesterol (Adult Treatment Panel III). According to the recent National Institutes of Health's, National Heart, Lung, and Blood Institute (NIH/NHLBI) publication; Expert Panel on Detection, Evaluation and Treatment of High Blood Cholesterol in Adults, Adult Treatment Panel III (ATP III) guidelines (2001), depending on the risk factor score, individuals with LDL-cholesterol levels from ≧100 to ≦130 mg/dL are recommended to initiate therapeutic lifestyle changes. Adults with LDL-cholesterol >130 mg/dL are recommended for intensive lifestyle therapy and an LDL-cholesterol-lowering drug therapy to achieve an LDL-cholesterol goal of <100 mg/dL. Patients with LDL levels >160 mg/dL should be considered for therapies with lipid-lowering drugs. The American Heart Association has estimated that over 100 million adults in the US exceed the optimal level of total cholesterol (American Heart Association web site).

The pathogenesis of atherosclerosis leading to the formation of unstable plaque has been recognized as one of the major causes of CHD (Lusis 2000). Recently, new understanding of the pathogenesis of atherosclerosis has placed emphasis on the inflammatory process as a key contributor to the formation of unstable plaque. The instability of the atherosclerotic plaque, rather than the degree of stenosis, is considered to be the primary culprit in the majority of myocardial infarctions (MI). This realization has led to the investigation of plaque biology and recognition that markers of inflammation may be useful as predictors of cardiovascular risk. Among the various candidate markers of inflammation, high sensitivity C-reactive protein (hs-CRP), a non-specific acute phase inflammatory marker, has received the most attention as a predictor of CHD (Ridker 2002).

Scientific Review

Lipoprotein Associated Phospholipase A2 (Lp-PLA2) is an enzymatically active 50 kD protein. Lp-PLA2 is a member of the phospholipase A2 family, and unlike most phospholipases, is Ca2+ independent. Lp-PLA2 has been previously identified and characterized by Tew et al. (1996), Caslake et al. (2000), and in WO 95/00649-A1, U.S. Pat. No. 5,981,252, U.S. Pat. No. 5,968,818, U.S. Pat. No. 6,177,257 (SmithKline Beecham) and WO 00/24910-A1, U.S. Pat. No. 5,532,152, U.S. Pat. No. 5,605,801, U.S. Pat. No. 5,641,669, U.S. Pat. No. 5,656,431, U.S. Pat. No. 5,698,403, U.S. Pat. No. 5,977,308 (ICOS Corporation) which are herein incorporated by reference. Lp-PLA2 is expressed by macrophages, with increased expression in atherosclerotic lesions (Hakkinin 1999). Lp-PLA2 circulates bound mainly to LDL, co-purifies with LDL, and is responsible for >95% of the phospholipase activity associated with LDL (Caslake 2000).

Oxidation of LDL in the endothelial space of the artery is considered a critical step in the development of atherosclerosis. Oxidized LDL, unlike native LDL, has been shown to be associated with a host of pro-inflammatory and pro-atherogenic activities, which can ultimately lead to atherosclerotic plaque formation (Glass 2001, Witztum 1994). Increasing evidence from basic research suggests that atherosclerosis has an inflammatory component and represents much more than simple accumulation of lipids in the vessel wall. The earliest manifestation of a lesion is the fatty streak, largely composed of lipid-laden macrophages known as foam cells. The precursors of these cells are circulating monocytes. The ensuing inflammatory response can further stimulate migration and proliferation of smooth muscle cells and monocytes to the site of injury, to form an intermediate lesion. As layers of macrophages and smooth muscle cells accumulate, a fibrous plaque is formed, which is characterized by a necrotic core composed of cellular debris, lipids, cholesterol, calcium salts and a fibrous cap of smooth muscle, collagen and proteoglycans. Gradual growth of this advanced lesion may eventually project into the arterial lumen, impeding the flow of blood. Further progression of atherosclerosis may lead to plaque rupture and subsequent thrombus formation, resulting in acute coronary syndromes such as unstable angina, MI or sudden ischemic death (Davies 2000, Libby 1996).

Lp-PLA2 plays a key role in the process of atherogenesis by hydrolyzing the sn-2 fatty acid of oxidatively modified LDL, resulting in the formation of lysophosphatidylcholine and oxidized free fatty acids (Macphee 1999). Both of these oxidized phospholipid products of Lp-PLA2 action are thought to contribute to the development and progression of atherosclerosis, by their ability to attract monocytes and contribute to foam cell formation, among other pro-inflammatory actions (Macphee 2001, Macphee 2002).

Clinical Review

Lp-PLA2 has been previously reported as a potential risk factor for CHD. The predictive value of plasma levels of Lp-PLA2 for CHD has been reported in a large, prospective case-control clinical trial involving 6,595 men with hypercholesterolemia, known as the West of Scotland Coronary Prevention Study (WOSCOPS) (Packard 2000). Lp-PLA2 was measured in 580 CHD cases (defined by non-fatal MI, death from CHD, or a revascularization procedure) and 1,160 matched controls. The results indicated that plasma levels of Lp-PLA2 were significantly associated with development of CHD events by univariate and multivariate analyses, with almost a doubling of the relative risk for CHD events for the highest quintile of Lp-PLA2 compared to the lowest quintile. The association of Lp-PLA2 with CHD was independent of traditional risk factors such as LDL-cholesterol and other variables. This study provided an encouraging preliminary indication of the clinical utility of Lp-PLA2 as a risk factor for CHD.

In a study of angiographically proven CHD, Lp-PLA2 was shown to be significantly associated with the extent of coronary stenosis (Caslake 2000).

In another study, in which only females were examined (n=246, 123 cases and 123 controls), baseline levels of Lp-PLA2 were higher among cases than controls (p=0.016), but was not significantly associated with CHD when adjusted for other cardiovascular risk factors. In this study, cases included 40% of women with stroke, 51% non-fatal myocardial infarction and 9% fatal CHD (Blake 2001).

### SUMMARY OF THE INVENTION

This invention is directed to a method for assessing risk of Coronary Vascular Disease (CVD) in a patient which comprises measuring levels of both Lipoprotein Associated Phospholipase A2 (Lp-PLA2) and C-reactive protein (CRP) in the patient, analyzing a risk associated with the level of CRP and a risk associated with the level of Lp-PLA2, and using the combined risks to assess the risk of CVD in the patient The invention is also directed to a method for assessing risk of Coronary Vascular Disease (CVD) in a patient with low to normal Low Density Lipoprotein Cholesterol (LDL) levels which comprises measuring levels of both LDL and Lipoprotein Associated Phospholipase A2 (Lp-PLA2) and in the patient, analyzing a risk associated with the level of LDL and a risk associated with the level of Lp-PLA2, and using the combined risks to assess the risk of CVD in the patient.

The invention is also directed to a method for treating a subject to reduce the risk of a Coronary Vascular Disease (CVD), comprising: selecting and administering to a subject who has above-normal levels of both C-reactive protein (CRP) and Lipoprotein Associated Phospholipase A2 (Lp-PLA2), a therapeutic molecule selected from the group consisting of statins, anti-inflammatory agents, Lp-PLA2 inhibitors or cholesterol reuptake inhibitors in an amount effective to lower the risk of the subject developing a future CVD.

Kits are also provided, for example, a kit for diagnosing a patient's susceptibility to Coronary Vascular Disease (CVD) comprising both a suitable assay for measuring Lipoprotein Associated Phospholipase A2 (Lp-PLA2) levels and a suitable assay for measuring C-reactive protein (CRP) levels wherein the levels of both CRP and Lp-PLA2 are determined. Alternatively, a kit for diagnosing a patient's susceptibility to Coronary Vascular Disease (CVD) comprising both a suitable assay for measuring Lipoprotein Associated Phospholipase A2 (Lp-PLA2) levels and a suitable assay for measuring Low Density lipoprotein Cholesterol (LDL) levels wherein the levels of both LDL and Lp-PLA2 are determined.

### BRIEF DESCRIPTION OF THE FIGURES

FIG. 1 shows Kaplan-Meier Survival Curves: Synergy of Lp-PLA2 and CRP. Patients categorized as below or above Lp-PLA2 or CRP medians (All LDL values).

FIG. 2 shows Kaplan-Meier Survival Curves: Synergy of Lp-PLA2 and CRP. Patients categorized as below or above Lp-PLA2 or CRP medians for subgroup with LDL<130 mg/dl.

FIG. 3 shows Kaplan-Meier Survival Curves: Synergy of Lp-PLA2 and CRP. Patients categorized as below or above Lp-PLA2 or CRP medians for subgroup with LDL<160 mg/dl.

FIG. 4 shows Kaplan-Meier Survival Curves: Synergy of Lp-PLA2 and CRP. Patients categorized in tertiles for both markers. ARIC Lp-PLA2 Study Population (n=1348).

FIG. 5 shows Kaplan-Meier Survival Curves: Synergy of Lp-PLA2 and CRP Patients categorized in tertiles for both markers. ARIC Lp-PLA2 Population with LDL<130 mg/dL (n=573).

FIG. 6 shows Kaplan-Meier Survival Curves: Synergy of Lp-PLA2 and CRP. Patients categorized in tertiles for both markers. ARIC LpPLA2 Population w/LDL>130 mg/dL (n=775).

FIG. 7 shows the association of Lp-PLA2 and CRP with incident CHD for all subjects.

FIG. 8 shows the association of Lp-PLA2 and CRP with incident CHD for LDL<130 mg/dL.

FIG. 9 shows association of Lp-PLA2 tertiles and CRP (1,3 as cut-offs) with incident CHD for LDL<130 mg/dL.

FIG. 10 shows the association of Lp-PLA2 tertiles for LDL<130 mg/dL for a variety of traditional risk factors. Abbreviations presented in the table, HT for hypertension, S for smoking, D for diabetes.

FIG. 11 shows the association of Lp-PLA2 tertiles for LDL<130 mg/dL for a variety of traditional risk factors. Abbreviations presented in the table, HT for hypertension, S for smoking, D for diabetes.

### DETAILED DESCRIPTION OF THE INVENTION

This invention is directed to a method for assessing risk of Coronary Vascular Disease (CVD) in a patient which comprises measuring levels of both Lipoprotein Associated Phospholipase A2 (Lp-PLA2) and C-reactive protein (CRP) in the patient, analyzing a risk associated with the level of CRP and a risk associated with the level of Lp-PLA2, and using the combined risks to assess the risk of CVD in the patient. The invention is also directed to a method for assessing risk of Coronary Vascular Disease (CVD) in a patient with low to normal Low Density Lipoprotein Cholesterol (LDL) levels which comprises measuring levels of both LDL and Lipoprotein Associated Phospholipase A2 (Lp-PLA2) and in the patient, analyzing a risk associated with the level of LDL and a risk associated with the level of Lp-PLA2, and using the combined risks to assess the risk of CVD in the patient In one embodiment the patient is diabetic. In another embodiment the patient is diabetic and hypertensive. In a further embodiment the patient is diabetic, hypertensive and smokes. In yet a further embodiment, the patient suffers from a metabolic disorder. In another embodiment, the Coronary Vascular Disease (CVD) is Coronary Heart Disease (CHD). In another embodiment the metabolic disorder includes but not limited to, obesity, overweight, diabetes, insulin resistance, anorexia, and cachexia. The invention may include measuring levels of low density lipoprotein cholesterol (LDL) and analyzing the respective levels of all three markers, LDL, CRP and Lp-PLA2, in combination so as to assess the risk of CVD in the patient.

In one embodiment, the respective levels of CRP and Lp-PLA2 are based on dividing a patient population dataset into high and low levels of each CRP and Lp-PLA2, such as using the median level, and a patient having both high CRP and high Lp-PLA2 levels is indicative of heightened risk of CVD. Alternatively, the patient dataset may be divided into tertiles, e.g., high, medium and low levels of each CRP and Lp-PLA2 and a patient having both high CRP and high Lp-PLA2 levels is indicative of heightened risk of CVD. In addition, LDL may also be measured in combination, and a patient having low LDL levels but having both high CRP and high Lp-PLA2 levels is indicative of heightened risk of CVD for the patient. Furthermore, a patient's additional risk of CVD may be determined using the ATP III guidelines. The measurements may be done simultaneously or sequentially.

The invention is also directed to a method for treating a subject to reduce the risk of a Coronary Vascular Disease (CVD), comprising: selecting and administering to a subject who has above-normal levels of both C-reactive protein (CRP) and Lipoprotein Associated Phospholipase A2 (Lp-PLA2), a therapeutic molecule selected from the group consisting of statin, anti-inflammatory agents, Lp-PLA2 inhibitors or cholesterol reuptake inhibitors in an amount effective to lower the risk of the subject developing a future CVD. Alternatively, the invention is directed to a method for treating a subject to reduce the risk of a Coronary Vascular Disease (CVD), comprising: selecting and administering to a subject who has both above-normal levels of Lipoprotein Associated Phospholipase A2 (Lp-PLA2) and low to normal levels of Low Density Lipoprotein Cholesterol (LDL) a therapeutic molecule selected from the group consisting of statins, Lp-PLA2 inhibitors or cholesterol reuptake inhibitors in an amount effective to lower the risk of the subject developing a future CVD.

Kits are also provided, for example, kit for diagnosing a patient's susceptibility to Coronary Vascular Disease (CVD) comprising both a suitable assay for measuring Lipoprotein Associated Phospholipase A2 (Lp-PLA2) levels and a suitable assay for measuring C-reactive protein (CRP) levels wherein the levels of both CRP and Lp-PLA2 are determined. Alternatively, a kit for diagnosing a patient's susceptibility to Coronary Vascular Disease (CVD) comprising both a suitable assay for measuring Lipoprotein Associated Phospholipase A2 (Lp-PLA2) levels and a suitable assay for measuring Low Density Lipoprotein Cholesterol (LDL) levels wherein the levels of both LDL and Lp-PLA2 are determined.

As used herein, the term “metabolic disorder” includes a disorder, disease or condition which is caused or characterized by an abnormal metabolism (i.e., the chemical changes in living cells by which energy is provided for vital processes and activities) in a subject. Metabolic disorders include diseases, disorders, or conditions associated with hyperglycemia or aberrant adipose cell (e.g., brown or white adipose cell) phenotype or function. Metabolic disorders can detrimentally affect cellular functions such as cellular proliferation, growth, differentiation, or migration, cellular regulation of homeostasis, inter- or intra-cellular communication; tissue function, such as liver function, renal function, or adipocyte function; systemic responses in an organism, such as hormonal responses (e.g., insulin response). Examples of metabolic disorders include obesity, diabetes, hyperphagia, endocrine abnormalities, triglyceride storage disease, Bardet-Biedl syndrome, Lawrence-Moon syndrome, Prader-Labhart-Willi syndrome, anorexia, and cachexia. Obesity is defined as a body mass index (BMI) of 30 kg/m.sup.2 or more (National Institute of Health, Clinical Guidelines on the Identification, Evaluation, and Treatment of Overweight and Obesity in Adults (1998)). However, the invention is also intended to include a disease, disorder, or condition that is characterized by a body mass index (BMI) of 25 kg/m2 or more, 26 kg/m2 or more, 27 kg/m.sup.2 or more, 28 kg/m.sup.2 or more, 29 kg/m.sup.2 or more, 29.5 kg/m.sup.2 or more, or 29.9 kg/m.sup.2 or more, all of which are typically referred to as overweight (National Institute of Health, Clinical Guidelines on the Identification, Evaluation, and Treatment of Overweight and Obesity in Adults (1998)).

Agents for reducing the risk of a Coronary Vascular Disorder include those selected from the group consisting of Lp-PLA2 inhibitors (Leach 2001), anti-inflammatory agents, anti-thrombotic agents, anti-platelet agents, fibrinolytic agents, lipid reducing agents, direct thrombin inhibitors, and glycoprotein IIb/IIIa receptor inhibitors and agents that bind to cellular adhesion molecules and inhibit the ability of white blood cells to attach to such molecules (e.g. anti-cellular adhesion molecule antibodies).

Anti-inflammatory agents include Alclofenac; Alclometasone Dipropionate; Algestone Acetonide; Alpha Arnylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit, Salcolex; Salnacedin; Salsalate; Salycilates; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide, Tetrydamine; Tiopinac; Tixocortol Pivalate; Tolmetin; Tolnetin Sodium; Triclonide; Triflumidate; Zidometacin; Glucocorticoids; Zomepirac Sodium.

Anti-thrombotic and/or fibrinolytic agents include Plasminogen (to plasmin via interactions of prekallikrein, kininogens, Factors XII, XIIIa, plasminogen proactivator, and tissue plasminogen activator[TPA]) Streptokinase; Urokinase: Anisoylated Plasminogen-Streptokinase Activator Complex; Pro-Urokinase; (Pro-UK); rTPA (alteplase or activase; r denotes recombinant), rPro-UK; Abbokinase; Eminase; Sreptase Anagrelide Hydrochloride; Bivalirudin; Dalteparin Sodium; Danaparoid Sodium; Dazoxiben Hydrochloride; Efegatran Sulfate; Enoxaparin Sodium; Ifetroban; Ifetroban Sodium; Tinzaparin Sodium; retaplase; Trifenagrel; Warfarin; Dextrans.

Anti-platelet agents include Clopridogrel; Sulfinpyrazone; Aspirin; Dipyridamole; Clofibrate; Pyridinol Carbamate; PGE; Glucagon; Antiserotonin drugs; Caffeine; Theophyllin Pentoxifyllin; Ticlopidine; Anagrelide. Lipid reducing agents include gemfibrozil, cholystyramine, colestipol, nicotinic acid, probucol lovastatin, fluvastatin, simvastatin, atorvastatin, pravastatin, cirivastatin (for statins, see Crouch 2000). Direct thrombin inhibitors include hirudin, hirugen, hirulog, agatroban, PPACK, thrombin aptamers. Glycoprotein IIb/IIIa receptor Inhibitors are both antibodies and non-antibodies, and include but are not limited to ReoPro (abcixamab), lamifiban, tirofiban. One preferred agent is aspirin.

Additional markers of systemic inflammation beyond CRP are well-known to those of ordinary skill in the art. It is preferred that the markers of systemic inflammation be selected from the group consisting of C-reactive protein, cytokines, and cellular adhesion molecules. Cytokines are well-known to those of ordinary skill in the art and include human interleukins 1-17. Cellular adhesion molecules are well-known to those of ordinary skill in the art and include integrins, ICAM-1, ICAM-3, BL-CAM, LFA-2, VCAM-1, NCAM, and PECAM. The preferred adhesion molecule is soluble intercellular adhesion molecule (sICAM-1).

The level of the markers of this invention may be obtained by a variety of recognized methods. Typically, the level is determined by measuring the level of the marker in a body fluid, for example, blood, lymph, saliva, urine and the like. The preferred body fluid is blood. The level can be determined by ELISA, or immunoassays or other conventional techniques for determining the presence of the marker. Conventional methods include sending samples of a patient's body fluid to a commercial laboratory for measurement. For the measurement of Lp-PLA2 enzymatic assays may also be used, see U.S. Pat. Nos. 5,981,252 or 5,880,273, the contents of which are hereby incorporated by reference into the subject application.

The invention also involves comparing the level of marker for the individual with a predetermined value. The predetermined value can take a variety of forms. It can be single cut-off value, such as a median or mean. It can be established based upon comparative groups, such as where the risk in one defined group is double the risk in another defined group. It can be a range, for example, where the tested population is divided equally (or unequally) into groups, e.g., tertiles, such as-a low-risk group, a medium-risk group and a high-risk group, or into quadrants, the lowest quadrant being individuals with the lowest risk and the highest quadrant being individuals with the highest risk.

There presently are commercial sources which produce reagents for assays for C-reactive protein. These include, but are not limited to, Abbott Pharmaceuticals (Abbott Park, Ill.), Dade Behring (Deerfield, Ill.) CalBiochem (San Diego, Calif.) and Behringwerke (Marburg, Germany). Commercial sources for inflammatory cytokine and cellular adhesion molecule measurements, include, but are not limited to, R&D Systems (Minneapolis, Minn.), Genzyme (Cambridge, Mass.) and Immunotech (Westbrook, Me.).

In preferred embodiments the invention provides novel kits or assays which are specific for, and have appropriate sensitivity with respect to, predetermined values selected on the basis of the present invention. The preferred kits, therefore, would differ from those presently commercially available, by including, for example, different cut-offs, different sensitivities at particular cut-offs as well as instructions or other printed material for characterizing risk based upon the outcome of the assay.

As discussed above the invention provides methods for evaluating the likelihood that an individual will benefit from treatment with an agent for reducing risk of a future cardiovascular disorder. This method has important implications for patient treatment and also for clinical development of new therapeutics. Physicians select therapeutic regimens for patient treatment based upon the expected net benefit to the patient. The net benefit is derived from the risk to benefit ratio. The present invention permits selection of individuals who are more likely to benefit by intervention, thereby aiding the physician in selecting a therapeutic regimen. This might include using drugs with a higher risk profile where the likelihood of expected benefit has increased. Likewise, clinical investigators desire to select for clinical trials a population with a high likelihood of obtaining a net benefit. The present invention can help clinical investigators select such individuals. It is expected that clinical investigators now will use the present invention for determining entry criteria for clinical trials.

An effective amount is a dosage of the therapeutic agent sufficient to provide a medically desirable result. The effective amount will vary with the particular condition being treated, the age and physical condition of the subject being treated, the severity of the condition, the duration of the treatment, the nature of the concurrent therapy (if any), the specific route of administration and the like factors within the knowledge and expertise of the health practitioner. For example, an effective amount can depend upon the degree to which an individual has abnormally elevated levels of markers of systemic information. It should be understood that the anti-inflammatory agents of the invention are used to prevent cardiovascular disorders, that is, they are used prophylactically in subjects at risk of developing a cardiovascular disorder. Thus, an effective amount is that amount which can lower the risk of, slow or perhaps prevent altogether the development of a cardiovascular disorder. When the agent is one that binds to cellular adhesion molecules and inhibits the ability of white blood cells to attach to such molecules, then the agent may be used prophylactically or may be used in acute circumstances, for example, post-myocardial infarction or post-angioplasty. It will be recognized when the agent is used in acute circumstances, it is used to prevent one or more medically undesirable results that typically flow from such adverse events. In the case of myocardial infarction, the agent can be used to limit injury to the cardiovascular tissue which develops as a result of the myocardial infarction and in the case of restenosis the agent can be used in amounts effective to inhibit, prevent or slow the reoccurrence of blockage. In either case, it is an amount sufficient to inhibit the infiltration of white blood cells and transmigration of white blood cells into the damaged tissue, which white blood cells can result in further damage and/or complications relating to the injury.

Generally, doses of active compounds would be from about 0.01 mg/kg per day to 1000 mg/kg per day. It is expected that doses ranging from 50-500 mg/kg will be suitable, preferably orally and in one or several administrations per day. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

When administered, the pharmaceutical preparations of the invention are applied in pharmaceutically-acceptable amounts and in pharmaceutically-acceptably compositions. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The anti-inflammatory agents, anti-Lp-PLA2 agents or statins may be combined, optionally, with a pharmaceutically-acceptable carrier. The term “pharmaceutically-acceptable carrier” as used herein means one or more compatible solid or liquid filler, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt. The pharmaceutical compositions also may contain, optionally, suitable preservatives, such as: benzalkonium chloride; chlorobutanol; parabens and thimerosal.

Compositions suitable for parenteral administration conveniently comprise a sterile aqueous preparation of the anti-inflammatory agent, which is preferably isotonic with the blood of the recipient. This aqueous preparation may be formulated according to known methods using suitable dispersing or wetting agents and suspending agents The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

A variety of administration routes are available. The particular mode selected will depend, of course, upon the particular drug selected, the severity of the condition being treated and the dosage required for therapeutic efficacy. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, interdermal, or parenteral routes. The term “parenteral” includes subcutaneous, intravenous, intramuscular, or infusion. Intravenous or intramuscular routes are not particularly suitable for long-term therapy and prophylaxis. They could, however, be preferred in emergency situations. Oral administration will be preferred for prophylactic treatment because of the convenience to the patient as well as the dosing schedule.

The pharmaceutical compositions may conveniently be presented in unit dosage form and may be prepared by any of the methods well-known in the art of pharmacy. All methods include the step of bringing the anti-inflammatory agent into association with a carrier which constitutes one or more accessory ingredients. In general, the compositions are prepared by uniformly and intimately bringing the anti-inflammatory agent into association with a liquid carrier, a finely divided solid carrier, or both, and then, if necessary, shaping the product.

Compositions suitable for oral administration may be presented as discrete units, such as capsules, tablets, lozenges, each containing a predetermined amount of the anti-inflammatory agent. Other compositions include suspensions in aqueous liquids or non-aqueous liquids such as a syrup, elixir or an emulsion.

Other delivery systems can include time-release, delayed release or sustained release delivery systems. Such systems can avoid repeated administrations of the anti-inflammatory agent, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyic acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; peptide based systems; wax coatings; compressed tablets using conventional binders and excipients; partially fused implants; and the like. Specific examples include, but are not limited to: (a) erosional systems in which the anti-inflammatory agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

### EXAMPLES

### Example 1

### Introduction

Lp-PLA2, LDL and CRP levels were studied using samples from the ARIC (Atherosclerosis Risk in Communities) sample set, ARIC database and a case-cohort design, in which a stratified random sample of the cohort was used, from which all controls were taken. In addition, all cases in the original cohort, whether in the random sample or not were used (Prentice 1986). The cohort random sample (CRS) was stratified by gender, age (≦54 vs. >54 yrs) and race (African-American/White).

The ARIC Study started recruitment in November of 1986 and took steps to enroll 16,000 individuals, ages 45-64. A total of 15,792 subjects were actually enrolled (Jackson 1997). At the time of enrollment, each participant received an extensive clinical examination. Thereafter, all participants were followed for the development of CHD annually by phone and by a clinic visit once every 3 years. At the second clinic visit, the extensive clinical examination was repeated, including physical, health and smoking status assessment, electrocardiogram, and ultrasound, and a blood sample was obtained from each subject during the clinical exam. The blood samples obtained from the second visit were used for this study.

The ARIC study and its cohort of samples are particularly relevant for testing the clinical utility of Lp-PLA2 as a risk predictor because of the diversity of the study population and the choice of the study endpoint (CHD event).

### Example 2

### Analysis Population

Because the baseline blood samples obtained from each subject upon entry to the ARIC Study have been depleted, the blood samples used herein consisted of those samples provided by each subject at the 2nd exam (scheduled for 1990-1992). Subjects included must have been free of heart disease prior to the time of the second blood collection (done at the time of the second exam). These subjects were followed for the development of CHD until 1998 or death, whichever occurred first. Of these subjects, 679 developed CHD during the follow-up period and NIH approved the use of these 679 cases, together with 801 stratified controls. These EDTA-plasma samples were stored at −70° C. since 1990. Information (including freeze/thaw history) concerning these samples was logged into the ARIC database and stored. To prevent any bias in the interpretation and reporting of Lp-PLA2 assay results, these plasma samples were tested for Lp-PLA2 levels in a blinded fashion by the Central Lipid Laboratory, Baylor College of Medicine. Samples were coded to mask any identifying information defining controls or cases. Results were stored, with the rest of the ARIC data, on the ARIC database at the University of North Carolina, Chapel Hill (UNC). 608 (45%) out of 1348 subjects were cases and 740 (55%) controls.

Table 2.1 summarized the subjects who were eligible from the original ARIC cohort.

TABLE 2.1
Original ARIC Cohort

| | Eligible from the |
|---|---|
| Stratum | Original Cohort |
| African-American female age | 801 |
| >54 | |
| African-American female age | 1246 |
| ≦54 | |
| African-American male age >54 | 470 |
| African-American male age ≦54 | 675 |
| White female age >54 | 2391 |
| White female age ≦54 | 2913 |
| White male age >54 | 2127 |
| White male age ≦54 | 2196 |
| Total | 12819 |

### Example 2.1

### Experimental Methods

Lp-PLA2 levels were measured using published methods (Dada 2002). The assay system utilized monoclonal anti-Lp-PLA2 antibody directed against Lp-PLA2 for solid phase immobilization on the microtiter stripwells. The test sample was first diluted with the sample diluent and incubated at 2-8° C. for 60 minutes. The diluted test sample was then allowed to react with the immobilized monoclonal antibody at 2-8° C. for 90 minutes. The wells were washed with distilled water to remove any unbound antigen. A second monoclonal anti-Lp-PLA2 antibody labeled with the enzyme horseradish peroxidase (HRP) was then added and reacted with the immobilized antigen at 2-8° C. for 60 minutes, resulting in the Lp-PLA2 molecules being captured between the solid phase and the enzyme-labeled antibodies. The wells were washed with distilled water to remove unbound labeled antibodies. The substrate, tetramethylbenzidine (TMB), was then added and incubated at 2-8° C. for 20 minutes resulting in the development of a blue color. Color development was stopped with the addition of Stop Solution (1N HCl), changing the color to yellow. The absorbance of the enzymatic turnover of the substrate was determined spectrophotometrically at 450 nm using a standard microplate reader and was directly proportional to the concentration of Lp-PLA2 present. A set of Lp-PLA2 calibrators is used to plot a standard curve of absorbance (y-axis) versus Lp-PLA2 concentration in ng/mL (x-axis) from which the Lp-PLA2 concentration in the test sample were determined. The concentration of Lp-PLA2 in each sample and control was then interpolated from the standard curve. This may be constructed using a point-to-point curve fit with appropriate calibration curve fitting software or manually using graph paper. Lp-PLA2 immunoassays are available from various clinical laboratories including Mayo Clinical Laboratories (Rochester, Minn.).

The CRP levels were measured using published Denka Seiken CRP assay (Roberts 2001). LDL and HDL were measured using standard methods.

### Exampl3 3

### Statistical Methods and Considerations

3.1 Outcome Variable (Cases)

Cases in this study were defined to be subjects who experienced any sign or symptom of coronary heart disease (CHD) subsequent to Visit 2 in the ARIC study. CHD was defined as: fatal or non-fatal myocardial infarction (MI), fatal CHD (not a definite fatal MI), coronary revascularization, or silent MI by ECG. Time to CHD was censored on Dec. 31, 1998, or at date of death for those who have died, or at date of last contact, for any subject lost to follow-up.

3.2 Analysis

Three Cox regression models were used to evaluate the association of Lp-PLA2 and CHD. The first included Lp-PLA2 alone in the model. The second adjusted for age, gender, and race (African-American and White). In the third multivariate model, adjustments were made for gender (female/male), age (continuous value at visit 2), race (Non-White/White), and other risk factors: LDL, HDL, high sensitivity C-reactive protein (CRP), current smoker (Y/N), diabetes (Y/N), blood pressure, and interaction of Lp-PLA2 and LDL. Since recent evidence from several prospective studies (Folsom 2002, Ridker 2000) indicates that C-reactive protein (CRP) was a marker of CHD, CRP was also considered in the model as a covariate. All analyses conducted using CRP excluded two subjects with missing CRP (i.e., a total of 1346 subjects were used). Relative risks were computed, as well as 95% confidence intervals (CIs) in relation to categories of Lp-PLA2 and other variables by use of weighted proportional hazards regression, accounting for the stratified random sampling and the case-cohort design by Barlow's method (Barlow 1994). The stratified random samples (CRS) represent the entire population of the four ARIC communities, including cases and controls. This method is designed to yield consistent estimates of the hazard ratios in Cox regression analysis, estimates that apply to the full cohort, not just to the selected sample.

Variables in the third model were discretized, with cutpoints taken from the NCEP risk-score models for cholesterol and the JNC-6 model for hypertension. The cutpoint for LDL was 130 mg/dL. The cutpoints for HDL were <40 mg/dL, 40 to <60 mg/dL and ≧60 mg/dL. The cutpoints for CRP were <1 mg/L, 1 to 3 mg/L, and >3 mg/L (Ridker 2000). The CRS was used to estimate tertiles (see Table 4.2.1 for the cutpoints).

### Example 4

### Results

4.1 Demographics and Baseline Risk Factors

Baseline demographics and other risk factors at Visit 2 of subjects in the study were summarized for cases, for controls, and for the total (see Tables 4.1 & 4.2). The distributions of gender, race, JNC-6 blood pressure, current smoking status (Y/N), and diabetes (Y/N) were significantly different between cases and controls (p<0.001, Chi-Square test). The distributions of age (≦54 or >54) and the continuous value of age were not substantially different between cases and controls.

Mean Lp-PLA2 levels were higher in the 608 cases than the 740 controls (427 ng/mL vs. 378 ng/mL, p<0.001, Wilcoxon rank sum test). Statistically significant differences in LDL, HDL, and CRP between cases and controls were also observed (p<0.001, Wilcoxon rank sum test).

TABLE 4.1
Demographics

| | Cases | Controls | Total | |
|---|---|---|---|---|
| Variables | (N = 608) | (N = 740) | (N = 1348) | p-value |
| Age (years) at | | | | 0.1431* |
| Visit 2 | | | | |
| Mean (SD) | 58.6 (5.44) | 58.1 (5.62) | 58.3 (5.54) | 0.2885** |
| Median | 59 | 58 | 59 | |
| Min-Max | 47-68 | 47-69 | 47-69 | |
| ≦54 | 168 (28%) | 224 (30%) | 392 (29%) | |
| >54 | 440 (72%) | 516 (70%) | 956 (71%) | |
| Gender | | | | <0.001* |
| Males | 412 (68%) | 381 (51%) | 793 (59%) | |
| Females | 196 (32%) | 359 (49%) | 555 (41%) | |
| Race | | | | <0.001* |
| White | 469 (77%) | 511 (69%) | 980 (73%) | |
| African-American | 139 (23%) | 229 (31%) | 368 (27%) | |

*Wilcoxon rank sum test
**Chi-Square test

TABLE 4.2
Risk Factors at Visit 2 (Unadjusted)

| | Cases | Controls | Total | |
|---|---|---|---|---|
| Variables | (N = 608) | (N = 740) | (N = 1348) | p-value |
| JNC-6 Blood Pressure | | | | <0.001* |
| JNC6BP1 | 202 (33.2%) | 337 (45.5%) | 539 (40.0%) | |
| JNC6BP2 | 118 (19.4%) | 152 (20.5%) | 270 (20.0%) | |
| JNC6BP3 | 114 (18.8%) | 102 (13.8%) | 216 (16.0%) | |
| JNC6BP4 | 122 (20.1%) | 108 (14.6%) | 230 (17.1%) | |
| JNC6BP5 | 52 (8.6%) | 41 (5.5%) | 93 (6.9%) | |
| Current Smoker | | | | <0.001*** |
| Yes | 177 (29.1%) | 152 (20.5%) | 329 (24.4%) | |
| No | 431 (70.9%) | 588 (79.5%) | 1019 (75.6%) | |
| Diabetes | | | | <0.001*** |
| Yes | 174 (28.6%) | 126 (17.0%) | 300 (22.3%) | |
| No | 434 (71.4%) | 614 (83.0%) | 1048 (77.7%) | |
| Lp-PLA2 (ng/mL) | | | | <0.001** |
| Mean (SD) | 426.9 (143.9) | 377.6 (130.2) | 399.8 (138.7) | |
| Median | 411.3 | 363.3 | 386.5 | |
| Min-Max | 87-990 | 77.5-948 | 77.5-990 | |
| LDL (mg/dL) | | | | <0.001** |
| Mean (SD) | 147.09 (38.32) | 132.26 (35.84) | 138.95 (37.70) | |
| Median | 144.80 | 129.90 | 136.20 | |
| Min-Max | 52.6-316.8 | 37.4-265.6 | 37.4-316.8 | |
| HDL (mg/dL) | | | | <0.001** |
| Mean (SD) | 42.19 (12.31) | 50.63 (17.20) | 46.82 (15.76) | |
| Median | 40 | 47 | 44 | |
| Min-Max | 16-98 | 18-129 | 16-129 | |
| CRP (mg/L) | | | | <0.001** |
| Mean (SD) | 3.880 (3.452) | 3.087 (3.311) | 3.444 (3.397) | |
| Median | 2.638 | 1.762 | 2.114 | |
| Min-Max | 0.065-15.605 | 0-17.948 | 0-17.948 | |

Note:
two subjects with missing CRP
*Cochran-Mantel-Haenszel test (Row Means Scores statistics)
**Wilcoxon rank sum test
***Chi-Square test

TABLE 4.3
Adjusted Means of Lp-PLA2, LDL, HDL, and CRP at Visit 2
(Adjusted for Age at Visit 2, Race, and Gender)

| | Cases | Controls | |
|---|---|---|---|
| Variables | (N = 608) | (N = 740) | p-value |
| Lp-PLA2 (ng/mL) | 404 | 372 | <0.001 |
| LDL (mg/dL) | 145.18 | 131.13 | <0.001 |
| HDL (mg/dL) | 45.54 | 51.24 | <0.001 |
| CRP (mg/L) | 4.051 | 3.041 | <0.001 |

*Using SUDAAN REGRESS procedure to conduct ANCOVA to account for the weighted analysis

Adjusted means of Lp-PLA2, LDL, HDL, and CRP are also presented in Table 4.3 for cases versus controls using ANCOVA (adjusted for age at Visit 2, gender, race) to account for the weighted analysis. The differences in adjusted means of Lp-PLA2, LDL, HDL, and CRP between cases and non-cases were statistically significant (p<0.001).

In addition, Lp-PLA2 was positively correlated with LDL (r=0.36, p<0.001) and negatively correlated with HDL (r=−0.33, p<0.001).

4.2 Selection of Lp-PLA2 Cutpoints

Since no definitive accepted cutpoints of Lp-PLA2 for the analysis are available in the literature to date, possible analytic cutpoints of Lp-PLA2 were explored based on the current data. After an evaluation of several cutpoints of Lp-PLA2, the analysis based on the tertiles was selected as the most appropriate. The results of analysis using Lp-PLA2 tertiles are summarized in the next sections.

TABLE 4.2.1
Tertiles of Lp-PLA2

| | Weighted Cutpoints (ng/mL) for |
|---|---|
| Tertiles | Lp-PLA2 |
| 33% | 311.0 |
| 67% | 422.0 |

4.3 Main Cox Regression Models

Model 1: Lp-PLA2 alone

Model 2: Lp-PLA2 adjusted for demographics including age (acontinuous value of age was used in all tested models), race, and gender

Model 3: Lp-PLA2 adjusted for demographics, diabetes, LDL (using high and low based on 130 mg/dL), HDL, CRP, current smoking status, blood pressure, and interaction of LDL and Lp-PLA2

Table 4.4 summarized the results of the three Cox regression models. In Model 1, with Lp-PLA2 alone, Lp-PLA2 was a significant predictor of time to CHD with a risk ratio (RR) of 2.50 (95% CI 1.89-3.31, p<0.001) for the 3^{rd} Lp-PLA2 tertile vs. the 1^{st} tertile and RR=1.49 for the 2^{nd} tertile vs. 1^{st} tertile (95% CI 1.11-1.99, p=0.008). Lp-PLA2 remained as a significant predictor of CHD with a risk ratio (RR) of 1.76 (95% CI 1.32-2.36, p<0.001) for the highest tertile vs. the lowest tertile in Model 2, adjusted for demographics (age, race, and gender).

The interaction between Lp-PLA2 and LDL (high or low based on 130 mg/dL, approximately the median of LDL in the CRS) was significant (p=0.002), i.e., there was a significant difference in the association of Lp-PLA2 with time to CHD between high LDL (LDL≧130 mg/dL) and low LDL (LDL<130 mg/dL) subgroups. In Model 3, with the interaction of LDL and Lp-PLA2, in the presence of demographic variables and other risk factors (LDL, HDL, current smoking status, blood pressure, diabetes, and CRP) as covariates, Lp-PLA2 was statistically significantly associated with CHD (p=0.003, RR=2.12 with 95% CI 1.29-3.48 for 3^{rd} tertile vs. 1^{st} tertile; p=0.029, RR=1.71 with 95% CI 1.06-2.75 for 2^{nd} tertile vs. 1^{st} tertile; see Table 4.4).

In conclusion, Lp-PLA2 was a statistically significant predictor of time to CHD, even after adjustment for all other prognostic factors (statistically adjusted for age, race, gender, current smoking status, blood pressure, diabetes, CRP, LDL, HDL, and Lp-PLA2-LDL interaction).

TABLE 4.4
Results of Cox Regression Models

| | Lp-PLA2 | | |
|---|---|---|---|
| Model | Levels* | p-value | Risk Ratio (95% CI) |
| 1 | 2T | p = 0.008 | 1.49 (1.11-1.99) |
| | 3T | p < 0.001 | 2.50 (1.89-3.31) |
| 2 | 2T | p = 0.154 | 1.24 (0.92-1.66) |
| | 3T | p < 0.001 | 1.76 (1.32-2.36) |
| 3 | 2T | p = 0.029 | 1.71 (1.06-2.75) |
| | 3T | p = 0.003 | 2.12 (1.29-3.48) |

*2T: 2^{nd} tertile vs. 1^{st} tertile; 3T: 3^{rd} tertile vs. 1^{st} tertile

4.4 Kaplan-Meier Survival Curves

Median Analysis:

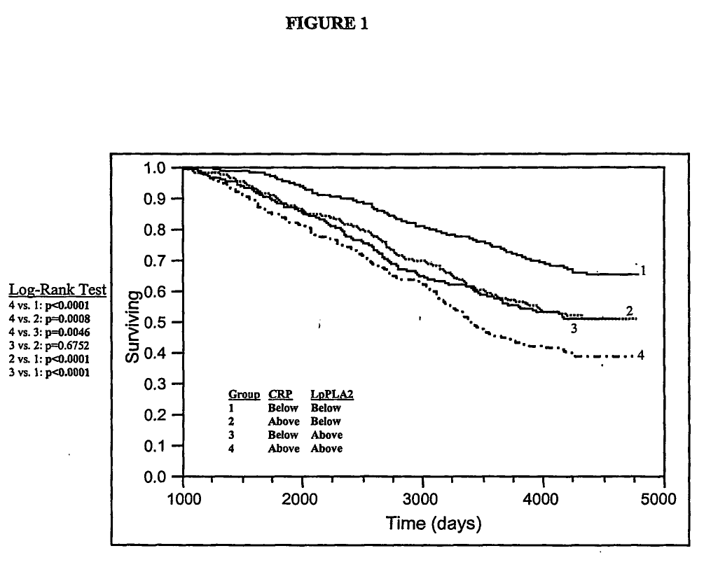

Kaplan-Meier survival curves demonstrate that use of medians Lp-PLA2 and CRP levels as cut points is statistically significant for the overall population, see FIG. 1. As indicated in FIG. 1, the time to CHD for the overall population was inversely related to Lp-PLA2 levels. The group with below the median levels for Lp-PLA2 and CRP had the longest time to CHD while the group with above the median levels of both Lp-PLA2 and CRP had the shortest time to CHD. The middle group, below median CRP, above median Lp-PLA2 and vis versa had an intermediate time to CHD. The difference was significant between these curves 4 (Lp-PLA2 and CRP) vs. 1, 2 or 3 with p<0.005 from log-rank test. The Log-Rank Test results were as follows: 4 vs. 1: p<0.0001; 4 vs. 2: p=0.0008; 4 vs. 3: p=0.0046; 3 vs. 2: p=0.6752; 2 vs. 1: p<0.0001; and 3 vs. 1: p<0.0001. The results in bold were statistically significant (see below).

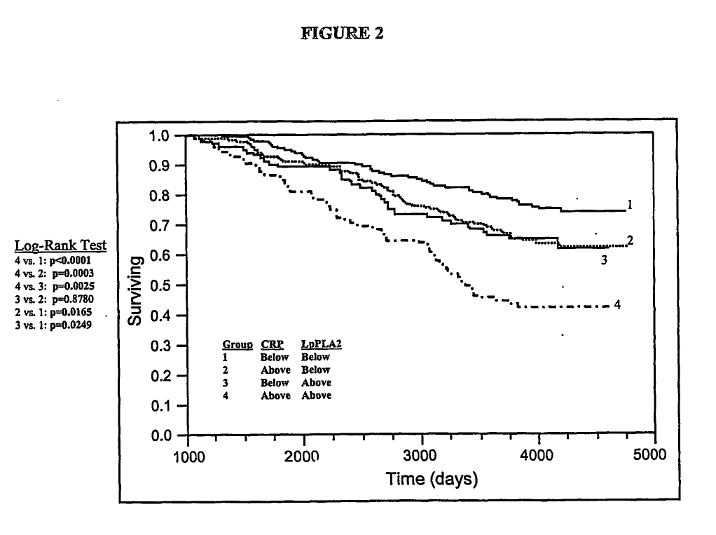

FIG. 2 shows similar Kaplan-Meier curves based on above and below the median Lp-PLA2 and CRP for patients with LDL<130 mg/dL The Log-Rank Test results were as follows: 4 vs. 1: p<0.0001; 4 vs. 2: p=0.0003; 4 vs. 3: p=0.0025; 3 vs. 2: p=0.8780; 2 vs. 1: p=0.0165; 3 vs. 1: p=0.0249.

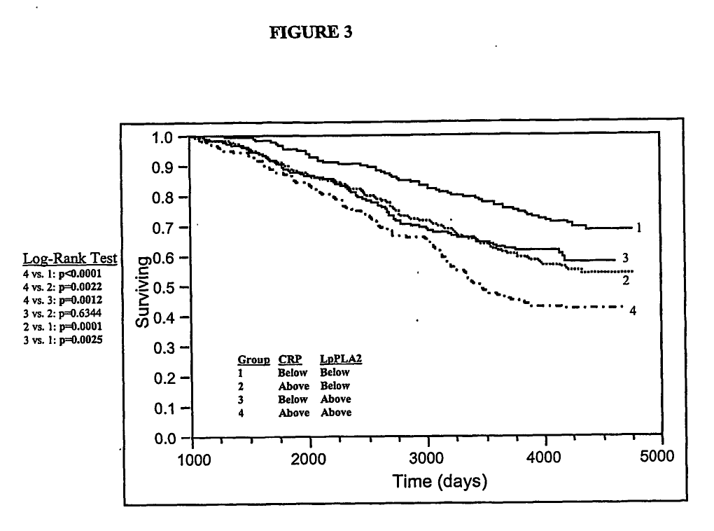

FIG. 3 shows Kaplan-Meier curves based on above and below the median Lp-PLA2 and CRP for patients with LDL<160 mg/dL. The Log-Rank Test results were as follows: 4 vs. 1: p<0.0001; 4 vs. 2: p=0.0022; 4 vs. 3: p=0.0012; 3 vs. 2: p=0.6344; 2 vs. 1: p=0.0001; 3 vs. 1: p=0.0025.

Tertile Analysis:

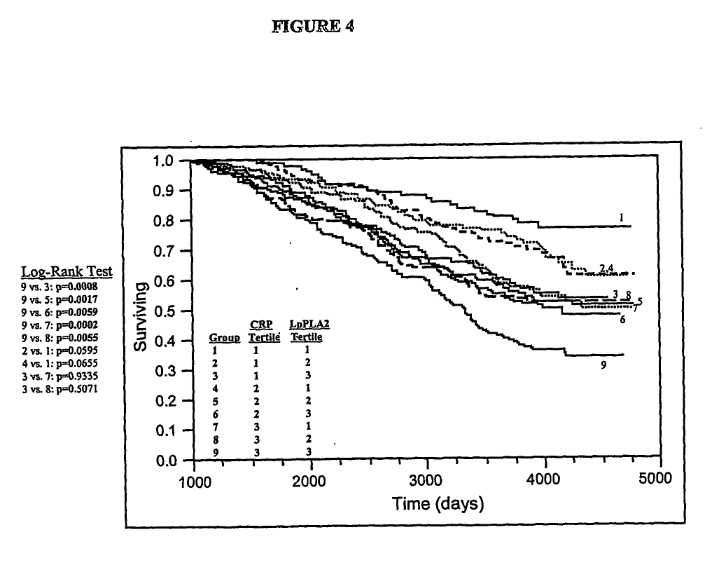

Kaplan-Meier survival curves are also presented by Lp-PLA2 and CRP tertiles for the overall population. The group with the lowest tertiles of both Lp-PLA2 and CRP had the longest time to CHD while the group with the highest tertiles of both Lp-PLA2 and CRP had the shortest time to CHD. The middle tertiles for Lp-PLA2 and CRP had an intermediate time to CHD. Specifically, Table 3.1 shows the cut points for the Lp-PLA2 analysis. Table 4.5 below shows the data underlying the Kaplan-Meier curve. The results are shown in FIG. 4. The Log-Rank Test results were as follows: 9 vs. 3: p=0.0008; 9 vs. 5: p=0.0017; 9 vs. 6: p=0.0059; 9 vs. 7: p=0.0002; 9 vs. 8: p=0.0055; 2 vs. 1: p=0.0595; 4 vs. 1: p=0.0655; 3 vs. 7: p=0.9335; 3 vs. 8: p=0.5071

TABLE 4.5
ARIC Lp-PLA2 Study Population (n = 1348)

| Synergy | CRP | Lp-PLA2 | n, Cases | n, Controls |
|---|---|---|---|---|
| Group # | Tertile | Tertile | (% of total) | (% of total) |
| 1 | 1 | 1 | 22 (1.6) | 76 (5.6) |
| 2 | 1 | 2 | 41 (3.0) | 81 (6.0) |
| 3 | 1 | 3 | 58 (4.3) | 74 (5.5) |
| 4 | 2 | 1 | 39 (2.9) | 80 (5.9) |
| 5 | 2 | 2 | 76 (5.6) | 86 (6.4) |
| 6 | 2 | 3 | 98 (7.3) | 102 (7.6) |
| 7 | 3 | 1 | 66 (4.9) | 82 (6.1) |
| 8 | 3 | 2 | 73 (5.4) | 82 (6.1) |
| 9 | 3 | 3 | 134 (9.9) | 78 (5.8) |

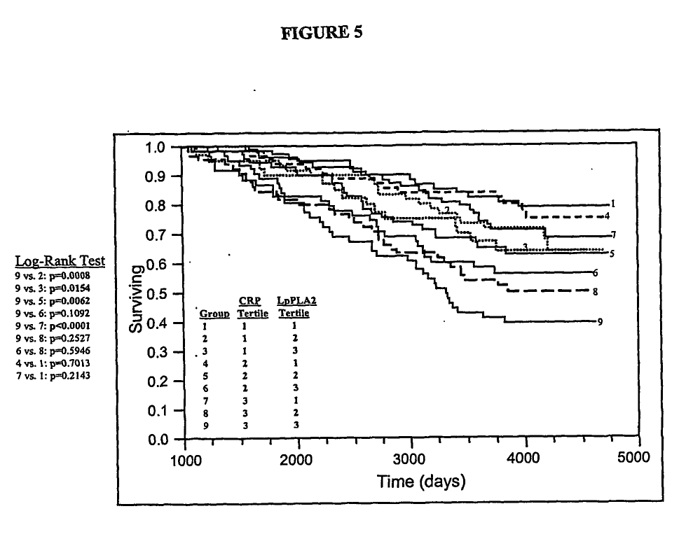

Table 4.6 shows the data for the tertile analysis of the patient population with LDL<130 mg/dL. The results are shown in FIG. 5. The Log-Rank Test results were as follows: 9 vs. 2: p=0.0008; 9 vs. 3: p=0.0154; 9 vs. 5: p=0.0062; 9 vs. 6: p=0.1092; 9 vs. 7: p<0.0001; 9 vs. 8: p=0.2527; 6 vs. 8: p=0.5946; 4 vs. 1: p=0.7013; and 7 vs. 1: p=0.2143.

TABLE 4.6
ARIC Lp-PLA2 Study Population w/LDL <130 mg/dL (n = 573)

| Synergy | CRP | Lp-PLA2 | n, Cases | n, Controls |
|---|---|---|---|---|
| Group # | Tertile | Tertile | (% of total) | (% of total) |
| 1 | 1 | 1 | 14 (2.4) | 55 (9.6) |
| 2 | 1 | 2 | 19 (3.3) | 42 (7.3) |
| 3 | 1 | 3 | 14 (2.4) | 26 (4.5) |
| 4 | 2 | 1 | 14 (2.4) | 50 (8.7) |
| 5 | 2 | 2 | 25 (4.4) | 45 (7.9) |
| 6 | 2 | 3 | 25 (4.4) | 34 (5.9) |
| 7 | 3 | 1 | 23 (4.0) | 59 (10.3) |
| 8 | 3 | 2 | 32 (5.6) | 33 (5.8) |
| 9 | 3 | 3 | 37 (6.5) | 26 (4.5) |

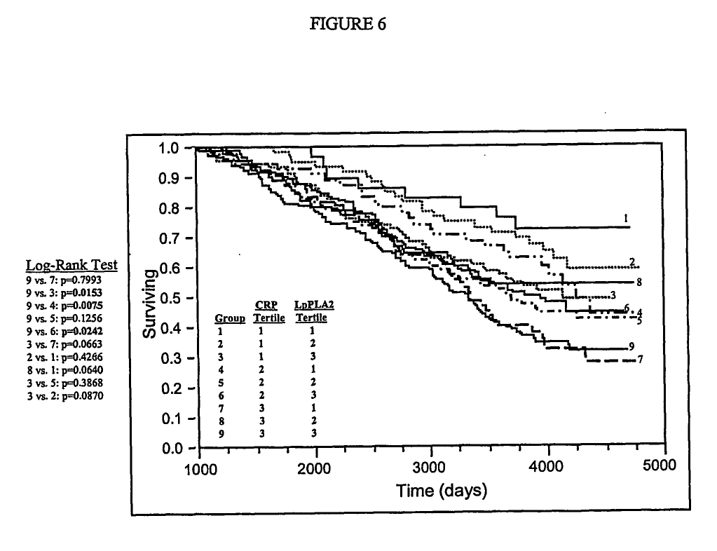

Table 4.7 shows the data for the tertile analysis of the patient population with LDL>130 mg/dL. The results are also shown in FIG. 6. The Log-Rank Test results were as follows: 9 vs. 7: p=0.7993; 9 vs. 3: p=0.0153; 9 vs. 4: p=0.0075; 9 vs. 5: p=0.1256; 9 vs. 6: p=0.0242; 3 vs. 7: p=0.0663; 2 vs. 1: p=0.4266; 8 vs. 1: p=0.0640; 3 vs. 5: p=0.3868; and 3 vs. 2: p=0.0870.

TABLE 4.7
ARIC Lp-PLA2 Study Population w/LDL >130 mg/dL (n = 775)

| Synergy | CRP | Lp-PLA2 | n, Cases | n, Controls |
|---|---|---|---|---|
| Group # | Tertile | Tertile | (% of total) | (% of total) |
| 1 | 1 | 1 | 8 (1.0) | 21 (2.7) |
| 2 | 1 | 2 | 22 (2.8) | 39 (5.0) |
| 3 | 1 | 3 | 44 (5.7) | 48 (6.2) |
| 4 | 2 | 1 | 25 (3.2) | 30 (3.9) |
| 5 | 2 | 2 | 51 (6.6) | 41 (5.3) |
| 6 | 2 | 3 | 73 (9.4) | 68 (8.8) |
| 7 | 3 | 1 | 43 (5.6) | 23 (3.0) |
| 8 | 3 | 2 | 41 (5.3) | 49 (6.3) |
| 9 | 3 | 3 | 97 (12.5) | 52 (6.7) |

4.5 LDL Subgroups

Table 4.8 summarizes the results of the three Cox regression models in the subgroup with LDL<130 mg/dL. In the model with Lp-PLA2 alone, Lp-PLA2 was a strong predictor of time to cardiac events with a risk ratio (RR) of 3.52 (95% CI 2.25-5.49, p<0.001) for the 3^{rd} Lp-PLA2 tertile vs. the 1^{st} tertile and RR=2.17 for the 2^{nd} tertile vs. 1^{st} tertile (95% CI 1.41-3.36, p=0.008). Lp-PLA2 remained a strong predictor of CHD with a risk ratio (RR) of 2.21 (95% CI 1.39-3.51, p<0.001) for the highest tertile vs. the lowest (RR=1.59, 95% CI 1.03-2.46, p=0.038) in the model adjusted for demographics (age, race, and gender). In those individuals with LDL<130 mg/dL, Lp-PLA2 was also a strong predictor in spite of adjustment for all other prognostic factors, and was more highly significant, with higher risk ratio, than CRP (p=0.012, RR=2.04 with 95% CI 1.17-3.55 for the Lp-PLA2 3^{rd} tertile vs. 1^{st} tertile compared to p=0.051, RR=1.73 for CRP>3 vs. CRP<1). This was not seen in the subgroup with LDL≧130 mg/dL.

More importantly, for those individuals with LDL<130 mg/dL, Lp-PLA2 is a particularly strong marker of CHD risk with approximately double risk comparing the highest to lowest tertiles of Lp-PLA2 in spite of adjustment for all other prognostic factors.

TABLE 4.8
Weighted Proportional Hazard Regression Models, For LDL<130 mg/dL

| | | | | | |
|---|---|---|---|---|---|
| | Regression | | | | |
| Factors | Coefficient | Standard Error | Risk Ratio | 95% CI | p-value |

Lp-PLA2 Alone

| | | | | | |
|---|---|---|---|---|---|
| Lp-PLA2 2^{nd} vs. 1^{st} | 0.78 | 0.22 | 2.17 | (1.41-3.36) | 0.000 |
| Lp-PLA2 3^{rd} vs. 1^{st} | 1.26 | 0.23 | 3.52 | (2.25-5.49) | 0.000 |

Lp-PLA2 Adjusted for Demographics

| | | | | | |
|---|---|---|---|---|---|
| Lp-PLA2 2^{nd} vs. 1^{st} | 0.46 | 0.22 | 1.59 | (1.03-2.46) | 0.038 |
| Lp-PLA2 3^{rd} vs. 1^{st} | 0.79 | 0.24 | 2.21 | (1.39-3.51) | 0.001 |

Lp-PLA2 Adjusted for Demographics and Other Risk Factors

| | | | | | |
|---|---|---|---|---|---|
| CRP 1-3 VS <1 | 0.06 | 0.27 | 1.06 | (0.63-1.79) | 0.818 |
| CRP >3 VS <1 | 0.55 | 0.28 | 1.73 | (1.00-3.00) | 0.051 |
| HDL <40 VS. >=60 | 1.06 | 0.33 | 2.89 | (1.52-5.49) | 0.001 |
| HDL 40-60 VS. >=60 | 0.31 | 0.31 | 1.37 | (0.74-2.53) | 0.320 |
| LPPL2T | 0.56 | 0.26 | 1.75 | (1.05-2.92) | 0.033 |
| LPPL3T | 0.71 | 0.28 | 2.04 | (1.17-3.55) | 0.012 |

Given the difference in the high LDL subgroup (LDL≧130) from the results for the overall, the Lp-PLA2 tertiles (derived from the CRS across all LDL levels) may not represent the prediction trend of Lp-PLA2 well in this subgroup. In order to understand the prediction of time to CHD using Lp-PLA2 in the high LDL subgroup (LDL≧130 mg/dL), further analyses were therefore conducted using separate, subgroup-specific cutpoints of Lp-PLA2.

4.6 Subgroup Analyses for LDL≧130 mg/dL

Analyses for the high LDL subgroup (LDL≧130) were conducted using varied cut-offs of Lp-PLA2 as follows (see Table 4.9):

Using weighted 40th and 80th percentiles of Lp-PLA2 based on the subjects with LDL≧130 in the study population

TABLE 4.9
Subgroup-specific Cutpoints of Lp-PLA2 for LDL ≧130 mg/dL
40^{th} and 80^{th} Percentiles Based on Study
Population with LDL >=130 mg/dL

| | Weighted Cutpoints (ng/mL) for Lp-PLA2 |
|---|---|
| 40% | 382.8 |
| 80% | 533.8 |

For the high LDL subgroup (LDL≧130 mg/dL), higher levels of Lp-PLA2 were associated with increased incidence of, and decreased time to, CHD, when subgroup-specific cutpoints were used (see Table 4.10).

TABLE 4.10
Weighted Proportional Hazard Regression Models,
For LDL ≧ 130 mg/dL LDL ≧ 30, Lp-PLA2,
40% = 382.8, 80% = 533.8 ng/mL

| | | RR | 95% CI | P value |
|---|---|---|---|---|
| Model 1 | 2T | 1.26 | 0.91-1.74 | 0.163 |
| | 3T | 2.34 | 1.56-3.50 | 0.000 |
| Model 2 | 2T | 1.05 | 0.76-1.45 | 0.787 |
| | 3T | 1.65 | 1.08-2.51 | 0.020 |
| Model 3 | 2T | 1.01 | 0.70-1.45 | 0.972 |
| | 3T | 1.52 | 0.96-2.40 | 0.074 |

4.7 Combined Risk of Lp-PLA2 and CRP

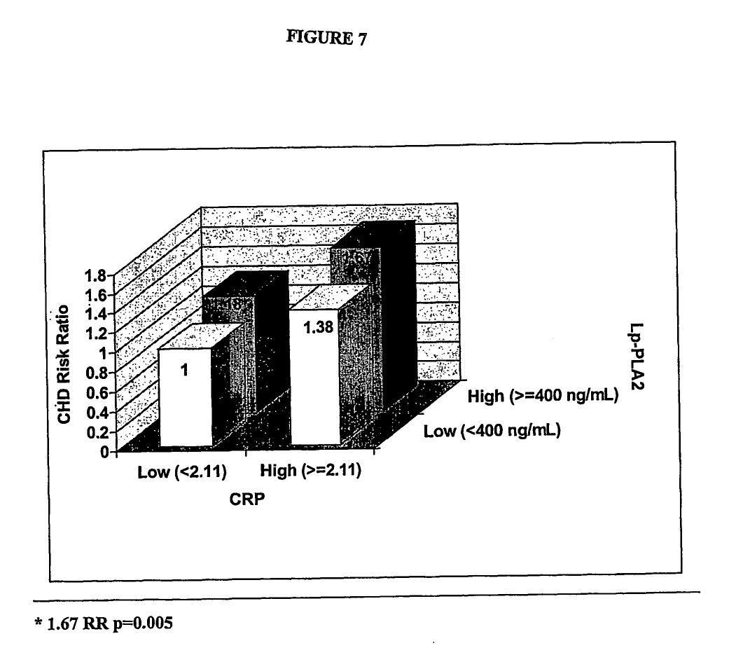

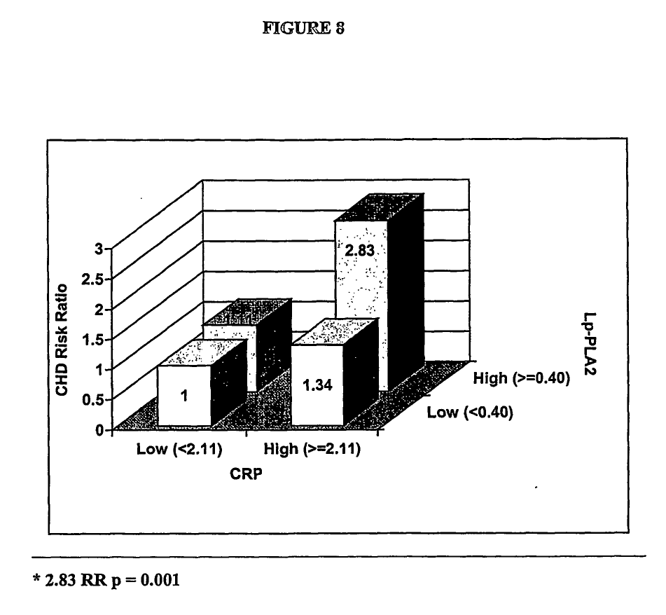

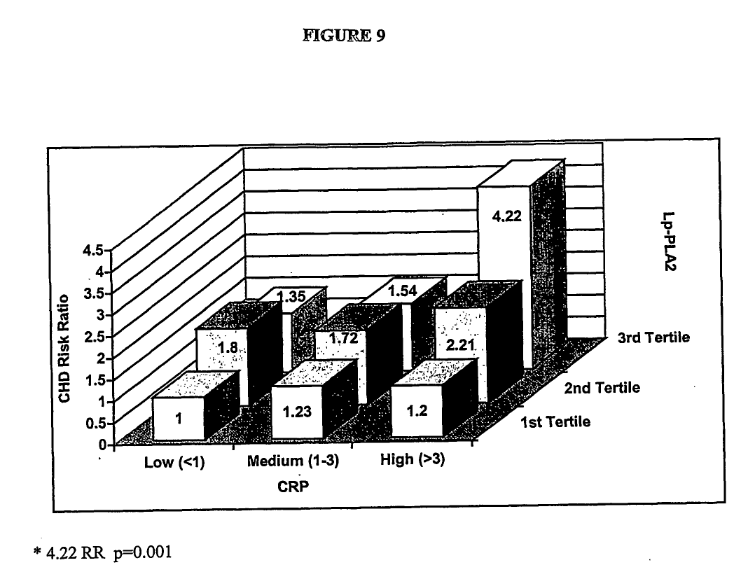

Tables 4.11-4.13 and FIGS. 7-9 present the combined risk of Lp-PLA2 and CRP for all subjects and for the low LDL subgroup (LDL<130 mg/dL). For individuals with low LDL, increased levels of both Lp-PLA2 and CRP corresponded with markedly increased risk for CHD (p=0.001, RR=4.22 with 95% CI (1.74-10.3), for Lp-PLA2 3rd tertile and CRP>3 vs. Lp-PLA2 1st tertile and CRP<1).

TABLE 4.11
Combined Risk of Lp-PLA2 and CRP Using Medians of Lp-PLA2 and
CRP as Cutpoints
For all Subjects (N = 1348)

| | | Stand- | | | |
|---|---|---|---|---|---|
| | Regression | ard | Risk | | p- |
| Factors | Coefficient | Error | Ratio | 95% CI | value |
| CRP_L/Lppla2_H | 0.17 | 0.18 | 1.18 | (0.83-1.68) | 0.346 |
| CRP_H/Lppla2_L | 0.33 | 0.18 | 1.38 | (0.97-1.97) | 0.069 |
| CRP_H/Lppla2_H | 0.51 | 0.18 | 1.67 | (1.17-2.39) | 0.005 |
| HDL <40 VS. >=60 | 1.02 | 0.21 | 2.76 | (1.82-4.21) | 0.000 |
| HDL 40-60 VS. | 0.47 | 0.20 | 1.59 | (1.08-2.35) | 0.019 |
| >=60 | | | | | |
| LDLHI | 0.56 | 0.13 | 1.76 | (1.36-2.27) | 0.000 |

TABLE 4.10
Combined Risk of Lp-PLA2 and CRP Using Medians of Lp-PLA2 and
CRP as Cutpoints
For LDL <130 mg/dL

| | | Stand- | | | |
|---|---|---|---|---|---|
| | Regression | ard | Risk | | p- |
| Factors | Coefficient | Error | Ratio | 95% CI | value |
| CRP_L/Lppla2_H | 0.11 | 0.30 | 1.11 | (0.62-2.00) | 0.724 |
| CRP_H/Lppla2_L | 0.29 | 0.28 | 1.34 | (0.78-2.31) | 0.287 |
| CRP_H/Lppla2_H | 1.04 | 0.30 | 2.83 | (1.57-5.10) | 0.001 |
| HDL <40 VS. | 1.11 | 0.33 | 3.03 | (1.59-5.76) | 0.001 |
| >=60 | | | | | |
| HDL 40-60 VS. | 0.37 | 0.32 | 1.45 | (0.78-2.69) | 0.242 |
| >=60 | | | | | |

TABLE 4.11
Combined Risk of Lp-PLA2 and CRP
Using Lp-PLA2 Tertiles and CRP Tertiles (1 and 3 ug/mL as cutpoints)
For LDL <130 mg/dL

| | Regression | Standard | Risk | | |
|---|---|---|---|---|---|
| Factors | Coefficient | Error | Ratio | 95% CI | p-value |
| CRPH_LPPL1 | 0.18 | 0.45 | 1.20 | (0.50-2.89) | 0.686 |
| CRPH_LPPL2 | 0.79 | 0.46 | 2.21 | (0.90-5.45) | 0.085 |
| CRPH_LPPL3 | 1.44 | 0.45 | 4.22 | (1.74-10.3) | 0.001 |
| CRPL_LPPL2 | 0.59 | 0.46 | 1.80 | (0.73-4.42) | 0.198 |
| CRPL_LPPL3 | 0.30 | 0.49 | 1.35 | (0.52-3.51) | 0.535 |
| CRPM_LPPL1 | 0.21 | 0.48 | 1.23 | (0.48-3.14) | 0.660 |
| CRPM_LPPL2 | 0.54 | 0.43 | 1.72 | (0.74-3.99) | 0.207 |
| CRPM_LPPL3 | 0.43 | 0.43 | 1.54 | (0.67-3.54) | 0.313 |
| HDL <40 VS. | 1.13 | 0.33 | 3.10 | (1.61-5.98) | 0.001 |
| >=60 | | | | | |
| HDL 40-60 | 0.36 | 0.32 | 1.43 | (0.77-2.66) | 0.259 |
| VS. >=60 | | | | | |

For the low LDL subgroup (LDL<130 mg/dL), higher levels of Lp-PLA2 were significantly associated with increased incidence of, and decreased time to, CHD. More importantly, for those individuals with LDL<130 mg/dL, Lp-PLA2 is a particularly strong marker of CHD risk with approximately double risk comparing the highest to lowest tertiles of Lp-PLA2 in spite of adjustment for all other prognostic factors. As the data above shows CRP and Lp-PLA2 are complimentary markers of CHD risk and patients with high levels of both CRP and Lp-PLA2 (whether by tertile or median analysis) show unusually high risk, even in the <130 LDL subgroup.

4.8 Combined Risk of Lp-PLA2 and Traditional Risk Factors

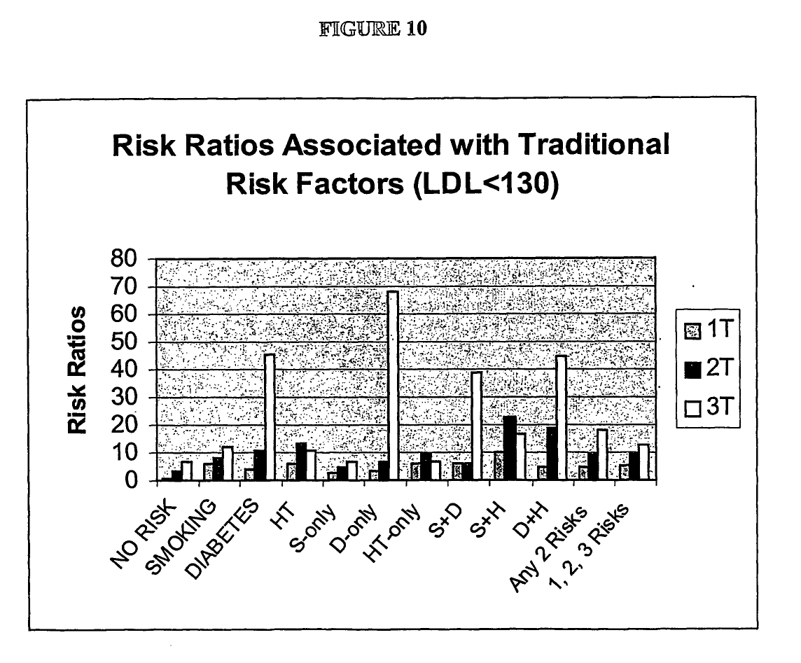

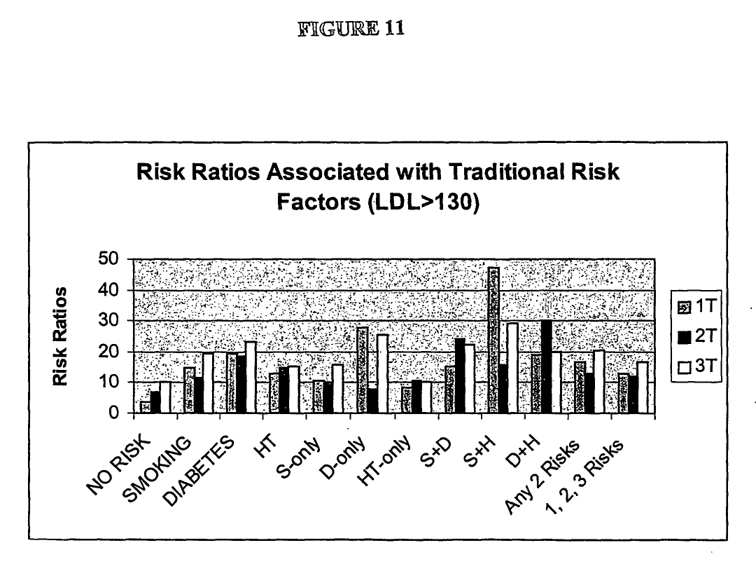

Cox regression analysis was performed on a variety of subpopulations with traditional risk factors. Specifically, hypertension, diabetes and smoking were examined either alone or in combination. The results show that the highest Lp-PLA2 tertile conferred a dramatic increase in risk for the diabetic subpopulation in the LDL<130 group. See FIGS. 10 and 11 and table 4.12 below.

TABLE 4.12
Risk Ratios for CHD Using the NO RISK Group (LDL <130, Lp-PLA2 <311
and No Smoking, Diabetes and Hypertension) as Reference:
LDL Subgroups: <130; Lp-PLA2 Cutpoints: 311/422

| | | LDL <130 mg/dL | | | |
|---|---|---|---|---|---|
| Lp-PLA2 | | | | | No. cases |
| cuts ng/mL | | 1T | 2T | 3T | No. controls |
| No Risk | Risk Ratio¹ | 1 | 3.1 | 6.8 | |
| | #CHD cases/total | 11/95 | 21/85 | 27/65 | Cases: 59 |
| | subjects in category | (11 + 84) | (21 + 64) | (27 + 38) | Controls: 186 |
| | cases + controls (%) | 11% | 24% | 41% | |
| Smoking | Risk Ratio¹ | 5.7 | 7.7 | 11.8 | |
| | #CHD cases/total | 17/54 | 17/37 | 21/43 | Cases: 55 |
| | subjects in category | (17 + 37) | (17 + 20) | (21 + 22) | Controls: 79 |
| | cases + controls (%) | 31% | 46% | 48% | |
| Diabetes | Risk Ratio¹ | 3.9 | 11.0 | 45.4 | |
| | #CHD cases/total | 12/44 | 21/41 | 25/34 | Cases: 58 |
| | subjects in category | (12 + 32) | (21 + 20) | (25 + 9) | Controls: 61 |
| | cases + controls (%) | 27% | 51% | 73% | |
| HT | Risk Ratio¹ | 6.3 | 13.6 | 10.8 | |
| | #CHD cases/total | 31/82 | 37/67 | 27/58 | Cases: 95 |
| | subjects in category | (31 + 51) | (37 + 30) | (27 + 31) | Controls: 112 |
| | cases + controls (%) | 37% | 55% | 46% | |
| Only S | Risk Ratio¹ | 2.7 | 4.8 | 6.8 | |
| | #CHD cases/total | 4/19 | 9/23 | 8/22 | Cases: 21 |
| | subjects in category | (4 + 15) | (9 + 14) | (8 + 14) | Controls: 43 |
| | cases + controls (%) | 21% | 39% | 36% | |
| Only D | Risk Ratio¹ | 3.5 | 6.8 | 68.1 | |
| | #CHD cases/total | 4/13 | 8/20 | 9/11 | Cases: 21 |
| | subjects in category | (4 + 9) | (8 + 12) | (9 + 2) | Controls: 23 |
| | cases + controls (%) | 30% | 40% | 81% | |
| Only H | Risk Ratio¹ | 6.3 | 9.2 | 6.4 | |
| | #CHD cases/total | 15/36 | 18/38 | 12/31 | Cases: 45 |
| | subjects in category | (15 + 21) | (18 + 20) | (12 + 19) | Controls: 60 |
| | cases + controls (%) | 41% | 47% | 38% | |
| S + D | Risk Ratio¹ | 5.8 | 6.3 | 38.7 | |
| | #CHD cases/total | 4/14 | 2/6 | 8/10 | Cases: 14 |
| | subjects in category | (4 + 10) | (2 + 4) | (8 + 2) | Controls: 16 |
| | cases + controls (%) | 28% | 33% | 80% | |
| S + H | Risk Ratio¹ | 9.9 | 22.7 | 17.0 | |
| | #CHD cases/total | 12/29 | 8/14 | 7/14 | Cases: 27 |
| | subjects in category | (12 + 17) | (8 + 6) | (7 + 7) | Controls: 30 |
| | cases + controls (%) | 41% | 57% | 50% | |
| D + H | Risk Ratio¹ | 4.4 | 18.6 | 44.4 | |
| | #CHD cases/total | 7/25 | 13/21 | 10/16 | Cases: 30 |
| | subjects in category | (7 + 18) | (13 + 8) | (10 + 6) | Controls: 32 |
| | cases + controls (%) | 28% | 61% | 62% | |
| D + H + S | Risk Ratio¹ | Not done due to insufficient sample size | | | |
| (n = 17) | #CHD cases/total | 3/8 | 2/6 | 2/3 | Cases: 7 |
| | subjects in category | (3 + 5) | (2 + 4) | (2 + 1) | Controls: 10 |
| | cases + controls (%) | 37% | 33% | 66% | |
| ≧1 Risk | Risk Ratio | 5.1 | 9.1 | 12.5 | |
| | #CHD cases/total | 40/120 | 105/194 | 50/98 | Cases: 195 |
| | subjects in category | (40 + 80) | (105 + 89) | (50 + 48) | Controls: 217 |
| | cases + controls (%) | 33% | 54% | 51% | |
| Any two | Risk Ratio¹ | 4.6 | 9.1 | 17.8 | |
| risks | #CHD cases/total | 25/84 | 36/72 | 38/67 | Cases: 99 |
| | subjects in category | (25 + 59) | (36 + 36) | (38 + 29) | Controls: 124 |
| | cases + controls (%) | 30% | 50% | 56% | |

¹1T “No Risk” is the reference

### REFERENCES

(2001). Executive Summary of The Third Report of The National Cholesterol Education Program (NCEP) Expert Panel on Detection, Evaluation, And Treatment of High Blood Cholesterol In Adults (Adult Treatment Panel III). JAMA 285(19): 2486-97.

Barlow, W. E. (1994). Robust variance estimation for the case-cohort design. Biometrics 50(4): 1064-72.

Blake, G. J., N. Dada, et al. (2001). A prospective evaluation of lipoprotein-associated phospholipase A(2) levels and the risk of future cardiovascular events in women. J Am Coll Cardiol 38(5): 1302-6.

Blake, G. J. and P. M. Ridker (2002). Inflammatory bio-markers and cardiovascular risk prediction. J Intern Med 252(4): 283-94.

Caslake, M. J., C. J. Packard, et al. (2000). Lipoprotein-associated phospholipase A(2), platelet-activating factor acetylhydrolase: a potential new risk factor for coronary artery disease. Atherosclerosis 150(2): 413-9.

Crouch, M. A. (2000). Effective use of statins to prevent coronary heart disease. American Family Physician 63 (2): 309-320.

Dada, N., N. W. Kim, et al. (2002). Lp-PLA2: an emerging biomarker of coronary heart disease. Expert Rev Mol Diagn 2(1): 17-22.

Davies, M. J. (2000). Pathophysiology of acute coronary syndromes. Heart 83:361-366.

Eaton, C. B., A. Monroe, et al. (1998). Cholesterol testing and management: a national comparison of family physicians, general internists, and cardiologists. J Am Board Fam Pract 11(3): 180-6.

Folsom, A. R., N. Aleksic, et al. (2002). C-reactive protein and incident coronary heart disease in the Atherosclerosis Risk In Communities (ARIC) study. Am Heart J 144(2): 233-8.

Glass, C. K. and J. L. Witztum (2001). Atherosclerosis: the road ahead. Cell 104(4): 503-16.

Hakkinen, T., J. S. Luoma, et al. (1999). Lipoprotein-associated phospholipase A(2), platelet-activating factor acetylhydrolase, is expressed by macrophages in human and rabbit atherosclerotic lesions. Arterioscler Thromb Vasc Biol 19(12): 2909-17.

Jackson R., L. E. Chambless, et al. (1997). Gender differences in ischaemic heart disease mortality and risk factors in 46 communities: an ecologic analysis. Cardiovasc Risk Factors 7: 43-54.

Leach, C. A., D. M. Hickey, et al. (2001). Lipoprotein-associated PLA2 inhibition—a novel, non-lipid lowering strategy for atherosclerosis therapy. Farmaco 56(1-2): 45-50.

Libby, P., Y. J. Geng, et al. (1996). Macrophages and atherosclerotic plaque stability. Curr Opin Lipidol 7(5): 330-5.

Lindahl, B., H. Toss, et al. (2000). Markers of myocardial damage and inflammation in relation to long-term mortality in unstable coronary artery disease. FRISC Study Group. N Engl J Med 343(16): 1139-47.

Lusis, A. J. (2000). Atherosclerosis. Nature 407(6801): 233-41.

Macphee, C. H. (2001). Lipoprotein-associated phospholipase A2: a potential new risk factor for coronary artery disease and a therapeutic target. Curr Opin Pharmacol 1(2): 121-5.

Macphee, C. H., K. E. Moores, et al. (1999). Lipoprotein-associated phospholipase A2, platelet-activating factor acetylhydrolase, generates two bioactive products during the oxidation of low-density lipoprotein: use of a novel inhibitor. Biochem J 338 (Pt 2): 479-87.

Macphee, C. H. and K. E. Suckling (2002). Lipoprotein-associated phospholipase A(2): a target directed at the atherosclerotic plaque. Expert Opin Ther Targets 6(3): 309-14.

Packard, C. J., D. S. O'Reilly, et al. (2000). Lipoprotein-associated phospholipase A2 as an independent predictor of coronary heart disease. West of Scotland Coronary Prevention Study Group. N Engl J Med 343(16): 1148-55.

Prentice, R. L. (1986). A case-cohort design for epidemiologic cohort studies and disease prevention trials. Biometrika 73(1):1-11.

Ridker, P. M., N. Rifai, et al. (2002). Comparison of C-reactive protein and low-density lipoprotein cholesterol levels in the prediction of first cardiovascular events. N Engl J Med 347(20): 1557-65.

Roberts, W. L., et al. (2001). Evaluation of nine automated high-sensitivity C-reactive protein methods: implications for clinical and epidemiological applications. Part 2. Clin Chem 2001;47:418-425.

Tew, D. G., et al. (1996). Purification, properties, sequencing, and cloning of a lipoprotein-associated, serine-dependent phospholipase involved in the oxidative modification of low-density lipoproteins. Arterioscler. Thromb. Vasc. Biol. 16:591-599.

Witztum, J. L. (1994). The oxidation hypothesis of atherosclerosis. Lancet 344(8925): 793-5.

### WEB SITES

American Heart Association, americanheart.org of the world wide web.

ARIC Study, cscc.unc.edu/aric/dirc.phtml of the world wide web

1. A method for assessing risk of Coronary Vascular Disease (CVD) in a patient which comprises measuring levels of both Lipoprotein Associated Phospholipase A2 (Lp-PLA2) and C-reactive protein (CRP) or Low Density Lipoprotein Cholesterol (LDL) in the patient, analyzing a risk associated with the level of CRP or LDL and a risk associated with the level of Lp-PLA2, and using the combined risks to assess the risk of CVD in the patient.

2. The method of claim 1 wherein the Coronary Vascular Disease (CVD) is Coronary Heart Disease (CHD).

3. The method of claim 1 which further comprises measuring levels of low density lipoprotein cholesterol (LDL) and analyzing the respective wherein levels of all three markers, LDL, CRP and Lp-PLA2 are analyzed, in combination, so as to assess the risk of CVD in the patient

4. The method of claim 1 wherein the measuring of CRP or LDL and Lp-PLA2 levels are done simultaneously.

5. The method of claim 1 wherein the measuring of CRP or LDL and Lp-PLA2 are done sequentially.

6. The method of claim 1 wherein levels of CRP and LP-PLA2 are analyzed and the respective levels of CRP and Lp-PLA2 are based on dividing a patient population dataset into high and low levels of each CRP and Lp-PLA2 and a patient having both high CRP and high Lp-PLA2 levels is indicative of heightened risk of CVD.

7. The method of claim 1 wherein levels of CRP and LP-PLA2 are analyzed and the respective levels of CRP and Lp-PLA2 are based on dividing a patient population dataset into high, medium and low levels of each CRP and Lp-PLA2 and a patient having both high CRP and high Lp-PLA2 levels is indicative of heightened risk of CVD.

8. The method of claim 3 wherein
(a) the respective levels of CRP and Lp-PLA2 are based on dividing a patient population dataset into high and low levels of each CRP and Lp-PLA2;
(b) the respective level of LDL is based on dividing the patient population dataset into high and low levels of LDL; and
(c) a patient having low LDL levels but having both high CRP and high Lp-PLA2 levels is indicative of heightened risk of CVD for the patient.

9. The method of claim 3 wherein
(a) the respective levels of CRP and Lp-PLA2 are based on dividing a patient population dataset into high, medium and low levels of each CRP and Lp-PLA2;
(b) the respective level of LDL is based on dividing the patient population dataset into high and low levels of LDL; and
(c) a patient having low LDL levels but having both high CRP and high Lp-PLA2 levels is indicative of heightened risk of CVD for the patient.

10. The method of claim 1 further comprising determining the patients risk of CVD using the ATP III guidelines.

11. The method claim 1 wherein the Lp-PLA2 levels are determined by measuring either Lp-PLA2 mass or Lp-PLA2 activity.

12-15. (canceled)

16. The method of claim 1 wherein levels of LDL and LP-PLA2 are analyzed and the levels of Lp-PLA2 are based on dividing a patient population dataset into high, medium and low levels of Lp-PLA2 and a patient having both high Lp-PLA2 levels and low to normal LDL is indicative of heightened risk of CVD.

17. (canceled)

18. The method claim 21 wherein the patient is both diabetic and hypertensive.

19. The method of claim 21 wherein the patient is diabetic, hypertensive and smokes.

20. The method of claim 1 wherein the patient suffers from a metabolic disorder.

21. The method of claim 20 where in the metabolic disorder is selected from the group consisting of, obesity, overweight, diabetes, insulin resistance, anorexia, and cachexia.

22-23. (canceled)

24. A method for treating a subject to reduce the risk of a Coronary Vascular Disease (CVD), comprising: selecting and administering to a subject who has above-normal levels of both C-reactive protein (CRP) and Lipoprotein Associated Phospholipase A2 (Lp-PLA2) or both above-normal levels of Lipoprotein Associated Phospholipase A2 (Lp-PLA2) and low to normal levels of Low Density Lipoprotein Cholesterol (LDL), a therapeutic molecule selected from the group consisting of statins, Lp-PLA2 inhibitors or cholesterol reuptake inhibitors in an amount effective to lower the risk of the subject developing a future CVD.

25. The method of claim 24 wherein the Coronary Vascular Disease (CVD) is Coronary Heart Disease (CHD).

26-29. (canceled)

30. A kit for diagnosing a patient's susceptibility to Coronary Vascular Disease (CVD) comprising both a suitable assay for measuring Lipoprotein Associated Phospholipase A2 (Lp-PLA2) levels and a suitable assay for measuring C-reactive protein (CRP) levels or Low Density Lipoprotein Cholesterol (LDL) levels wherein the levels of both CRP and Lp-PLA2 or both LDL and Lp-PLA2 are determined.

31. The kit of claim 30 wherein the Coronary Vascular Disease (CVD) is Coronary Heart Disease (CHD).

32. The kit of claim 30 wherein the suitable assay for measuring Lp-PLA2 levels measures either Lp-PLA2 mass or Lp-PLA2 activity assay.

33-35. (canceled)
